(19) United States
(12) Patent Application Publication
(10) Pub. No.: US 2002/0095122 A1
(43) Pub. Date: Jul. 18, 2002

(54) Accessing and deaccessing tool with elastomeric needle retainer & method

(76) Inventor: Terry M. Shaffer, (US)

Correspondence Address: JOHN J. CONNORS, Suite 220, 1600 DOVE ST., NEWPORT BEACH, CA, 92660, US

(21) Appl. No.: 09/851,357

(22) Filed: May 8, 2001

Related U.S. Application Data
(63) Continuation-in-part of application No. 09/346,828, filed on Jul. 2, 1999, now abandoned.

(51) Int. Cl. A61M 5/00

(52) U.S. Cl. 604/263

(57) ABSTRACT

A tool for accessing and deaccessing a needle includes an elongated body including a handle section and a guard section. There is a slot extending from an edge of the guard section into the guard section and terminating at an internal portion of the guard section. The handle section has a top surface with either retainer member with radial fingers or an elastomeric, plug-type retainer member. The tip of a needle is inserted into an exposed top end of the retainer member.

This application is a continuation-in-part of U.S. patent application Ser. No. 09/346,828, filed Jul. 2, 1999, entitled Accessing And Deaccessing Tool With Needle retainer And Method, now U.S. Pat. No. ______, which is incorporated herein by reference and made a part of this application.

### BACKGROUND OF THE INVENTION

In International Patent Application entitled Accessing and Deaccessing Tools and Methods, International Publication No. WO 97/43960, (herein International Patent Application) there are disclosed tools and methods for accessing and deaccessing a needle into or from a medical device implanted under the skin of a patient. These tools and methods are designed to reduce the risk of a nurse accidentally sticking herself or himself, or a patient, with the needle as it is being inserted into the implanted device or withdrawn from the implanted device.

Implanted medical devices, such as vascular access devices, are commonly used to allow medication to be administered to patients. One such implant device is sold under the trademark “BardPort” by C. R. Bard, Inc. These devices include a housing enclosing a chamber which has an inlet covered with a silicone or latex seal. An outlet in communication with the chamber allows fluid in the chamber to flow through the outlet into a tube which, typically, is inserted into the vein of a patient.

These devices are accessed periodically by a nurse who inserts a needle through the patient's skin overlying the device and then into and through the seal. The nurse palpates, or feels, the device through the skin overlying the implanted device, and presses downward to locate the position of the implanted device. While holding or pressing against the device through the skin with one hand, the nurse with the other hand inserts the needle through the skin, and into and through the seal. Once properly inserted through the skin, the tip of the needle penetrates the seal and is lodged within the chamber. A “click” sound can sometimes be heard when the tip of the needle touches the bottom of the chamber, or the nurse can feel the tip contact the bottom of the chamber. Typically, the needle penetrates a depth of from about ¼ inch to about 1 inch. Medication now flows through the needle into the chamber and then out the outlet through the tube into the vein of the patient. Sometimes, however, the nurse, while attempting to introduce the needle into the seal of the implanted device, accidentally sticks herself or himself with the needle. These accidental needle sticks occur while either accessing or deaccessing the implanted device with the needle.

Needle sticks occur most frequently while deaccessing the needle. The needle sometimes remains in the implanted medical device for several hours, and sometimes even for several days. These needles must be periodically flushed and removed from the device. The removal is accomplished by the nurse, with one hand, pressing against the skin overlying and around the device, and, with the other hand, grasping the needle and withdrawing it from the device. Frequently, there is an involuntary muscular recoil as the needle escapes from the implanted medical device as it is withdrawn. It is thought that this recoil is due to proprioceptive neuro-muscular activity. The recoil sometimes results in the nurse accidentally sticking a finger of the hand that is pressing against the skin adjacent to the implanted medical device. If the source patient has an infectious and or contagious disease such as disseminated TB, Hepatitis B or C, or is HIV positive or has AIDS, the nurse may contract the disease directly from this needle stick.

After such a needlestick, even if the source patient has no communicable and/or infectious disease or condition identified at the time of the needlestick, the nurse must undergo intensive and expensive follow-up testing intermittently for up to 1 year. The source patient must be tested, if they consent, for infectious or communicable disease as set forth in the OSHA regulations and CDC (Center for Disease Control) recommendations, under Employee Exposure to Bloodborne Pathogens. The nurse must also be counseled as to certain restrictions in his or her own lifestyle, particularly sex practices, until his or her own freedom from communicable/infectious disease or condition is determined. This places an incredible strain on the nurse's marital relationships and lifestyle. The partner often demands that the nurse quit nursing rather than face the risks.

Needle sticks also occur while accessing the implanted device. The problem of contracting an infectious, contagious disease also is sometimes encountered. For example, the needle, which is typically sterile initially, has in some reported instances completely penetrated the finger of the nurse and entered the body of the patient. The now contaminated needle can only be removed by withdrawing it from the patient's body into and through the nurse's finger, possibly infecting the nurse.

Recent guidelines promulgated by the CDC and OSHA prohibits medical procedures which require manipulating needles using both hands in the procedure, or any other technique that involves directing the point of a needle toward any part of the health care workers body. Under the current protocols for using the implanted device, the nurse's hand which secures the implanted device in place during accessing and deaccessing is always in direct line with the needle during accessing, and also in line with the needle tip during deaccessing when one considers the frequency of the known recoil phenomenon.

The problem of needle sticks while deaccessing the needle has been recognized by workers at the University Hospital in Antwerp, Belgium, who published an article in Infection Control and Hospital Epidemiology, Volume 14, No. 10 (October 1993). In this article it is suggested to use a tool, rather than the nurse's hand, to hold the implanted device during removal of the needle. The suggested tool includes a guard with a slot in it. The guard has a relatively small area. It appears to be less than 1 square inch, and it appears to be made of an opaque material. There is a short handle attached to the guard used to grasp the tool which does not permit the hand of the nurse to be located far enough away from the needle to insure avoiding needle sticks if a recoil occurs.

The accessing and deaccessing tool that is disclosed in the International Patent Application, has a retainer in a side wall of the tool into which a needle which has been extracted from an implanted device is inserted. In accordance with this invention, the accessing and deaccessing tool disclosed in this International Patent Application has been improved by relocating the position of the retainer and enhancing its ability to hold a needle inserted into it. The new position for the retainer is important to further reduce the risk of a nurse sticking herself or himself with an extracted needle upon inserting this needle into the retainer.

### SUMMARY OF THE INVENTION

This invention has several features, no single one of which is solely responsible for its desirable attributes. Without limiting the scope of this invention as expressed by the claims that follow, its more prominent features will now be discussed briefly. After considering this discussion, and particularly after reading the section entitled, “DETAILED DESCRIPTION OF THE PREFERRED EMBODIMENTS,” one will understand how the features of this invention provide its benefits, which include, but are not limited to, improved safety in inserting into a retainer in an accessing and deaccessing tool a needle extracted from an implanted device and the ability to dispose of needle being held in the retainer into bio-hazardous waste container without risk of needlestick, and an improved holding ability of a needle being held in such a retainer.

The first feature of the tool of this invention for accessing and deaccessing a needle is that it includes an elongated body including a handle section and a guard section. A slot extends from an edge of the guard section into the guard section and terminates at an internal portion of the guard section. The slot and the handle section are in substantial alignment with each other, both lying along a common longitudinal axis. The guard preferably has an area of 1.0 square inches or more, and the handle has a length of 3 inches or more. In accordance with this invention, the handle section has in a top surface a retainer member with a cavity therein into which the tip of a needle withdrawn from an implanted device is inserted. It is important that the retainer member be in the top surface to minimize the likelihood of needle sticks when inserting the extracted needle into retainer member. Because of the location of the retainer in the top surface of the tool and the orientation of the cavity, the likelihood of a nurse sticking herself or himself as the needle is inserted into the retainer is substantially reduced.

The second feature is that the cavity which receives the needle is elongated so that it receives the tip of the needle and its adjacent shank, approximately from about 0 to about 100% percent of the distal end of the needle. Preferable, the cavity is defined by a cylindrical wall terminating in a bottom section that tapers inwardly so that this bottom section has a substantially conical configuration. The diameter of the cavity is from about ⅛ to about ½ inch and its total length is about ⅛ to about 1½ inch and the length of the bottom section is about ⅛ to about ½ inch. The cavity is at an about 90 degrees or less with respect to the guard section, so that the tip of the needle is directed away from the body of the nurse. The position of the cavity and its orientation reduces the likelihood that a nurse will be stuck with as needle as she or he inserts the tip of the needle into the cavity. As the nurse inserts the needle into the retainer using one hand to hold the needle and the other hand to gripping the handle, she or he naturally points the tip of the needle away from her or his body. The handle section has a marker indicating that a hand of the nurse should be behind this marker. The marker is located to position the hand of the nurse at least 2.5 inches away from the needle when the tool is used to access or deaccess a needle in the implanted device. Preferably, the marker comprises a raised thumb lever, but other markers might be used as discussed in the International Patent Application.

The third feature is that the guard has an underside with a concave section having a substantially circular rim and the slot that extends radially outward from the center of the concave section to the edge of the guard section. The concave section has a depth of from 1/16 to 3/16 inch, and the rim has a diameter or width of from 1/16 to ½ inch. When the guard is circular, preferably the slot terminates in the guard section as an enlarged open portion. The slot has a width of from 1/16 to ¼ inch, and the enlarged open portion has an area in excess of 0.0036 square inch.

In one embodiment, the retainer member has a plurality of radial fingers projecting into the cavity. These radial fingers are arranged in a plurality of sets that overlie each other. Preferably, there are four fingers in each set, and each finger in each set is spaced apart to provide a cross-shaped opening between the fingers in each set. The fingers are substantially at a right angle with respect to the longitudinal axis of the cavity, and preferably they are disposed in a helical configuration along the cylindrical wall of the cavity. Consequently, they are offset with respect to each other, however, the fingers may be aligned. The inward ends of the fingers of each set of four fingers define an open central portion that has an area that is less then the cross-sectional area of the shank of the needle, typically having an area from about 1/64 to about ¼ square inch. The open central portions of each set are in registration with each other, but the sets of fingers are slightly unaligned and not in exact registration. These aligned open central portions provide an elongated central open space along the longitudinal axis of the cavity that is the ideal target into which the tip of the needle is forced upon inserting the needle into the cavity. The tip of the insert needle bottoms out and has its tip lodged in the apex of the conical bottom section. The fingers, being flexible, will bend upon engaging the tip of the needle, even if the user does not hit this ideal target, the needle is nevertheless firmly held within the retainer. Moreover, because of the flexibility of the fingers, this improves the grip of the fingers on the tip of the needle inserted into the retainer.

In another embodiment, there is a plug-type elastomeric retainer member seated within the cavity. Preferably, the plug-type retainer memebr is cylindrically shaped and made of an elastomeric material such as polyurethane. Preferably, this elastomeric material has a durometer hardness from about 45 to about 65. The plug-type retainer member has a top end and is seated within an elongated cavity with an open mouth so that the top end is recessed with respect to the open mouth.

This invention also includes a method of manually removing a needle from a medical device implanted under the skin of a patient. This method comprises:

grasping with one hand a handle section of a tool having a guard section, and an slot extending from an edge of the guard section into the guard section and terminating at an internal portion of the guard section, said handle section having a top surface with a retainer member therein into which the tip of a needle may be inserted,

sliding the slot along the needle to bring the guard section into an overlying relationship with the implanted medical device,

maintaining said one hand a minimum distance away from the needle of at least 2.5 inches,

with the other hand withdrawing said needle free of the implanted medical device, and

inserting said withdrawn needle into the retainer.

The retainer member may be either the finger-type or the plug-type made of an elastomeric material.

### DESCRIPTION OF THE DRAWING

The preferred embodiment of this invention, illustrating all its features, will now be discussed in detail. This embodiment depicts the novel and non-obvious accessing and deaccessing and method of this invention as shown in the accompanying drawing, which is for illustrative purposes only. This drawing includes the following FIGS. (FIGS.), with like numerals indicating like parts:

FIG. 1 is a perspective view of the tool of this invention looking at the topside of the tool.

FIG. 2 is a perspective view of the tool shown in FIG. 1 looking at the underside of the tool.

FIG. 3A is a plan view taken along line 3A-3A of FIG. 1 looking at the top surface of the handle into the retainer.

FIG. 3B is an enlarged, fragmentary view taken along line 3B of FIG. 3A.

FIG. 4A is a perspective, fragmentary view with sections broken away.

FIG. 4B is a cross-sectional view taken along line 4B-4B of FIG. 4A.

FIG. 4C is a cross-sectional view similar to that shown in FIG. 4B, except a needle is shown lodged in the retainer.

FIG. 5 is a cross-sectional view taken along line 5-5 of FIG. 4B.

FIG. 6 is a cross-sectional view taken along line 6-6 of FIG. 4B.

FIG. 7 is a cross-sectional view taken along line 7-7 of FIG. 4B.

FIG. 8 is a perspective, fragmentary view with sections broken away, of an alternate embodiment of this invention.

FIG. 9 is a cross-sectional view taken along line 9-9 of FIG. 8.

### DETAILED DESCRIPTION OF THE PREFERRED EMBODIMENTS

First Embodiment:

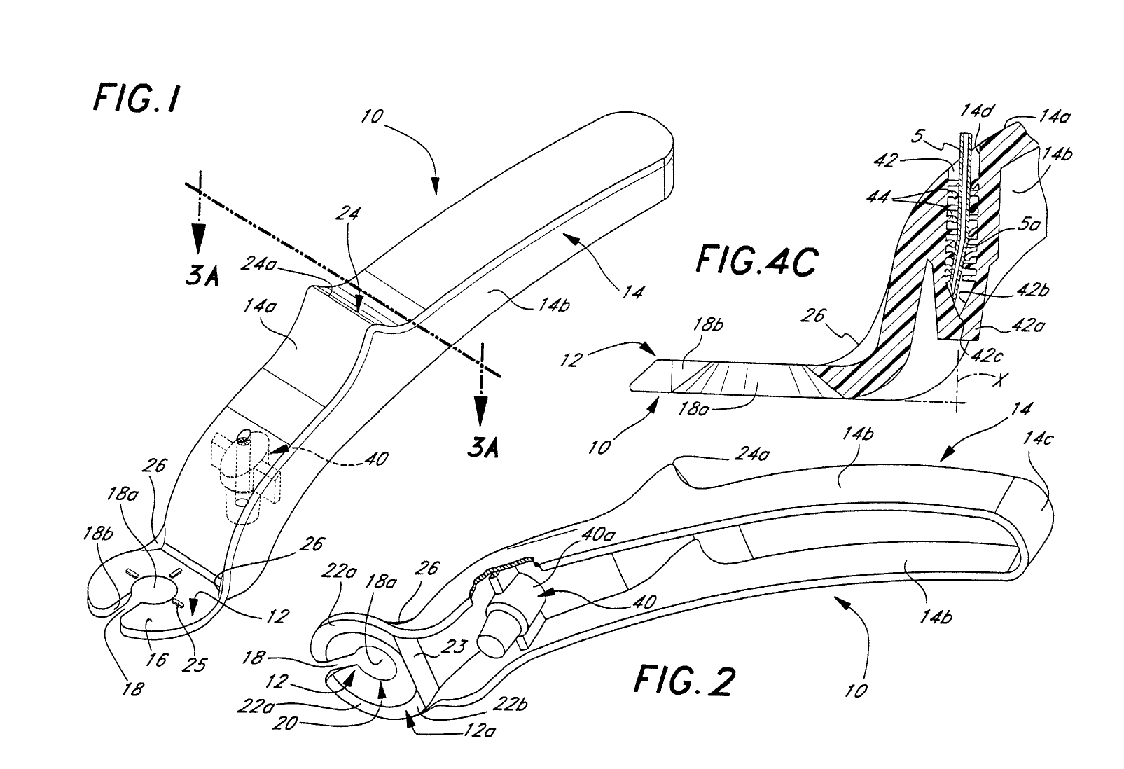

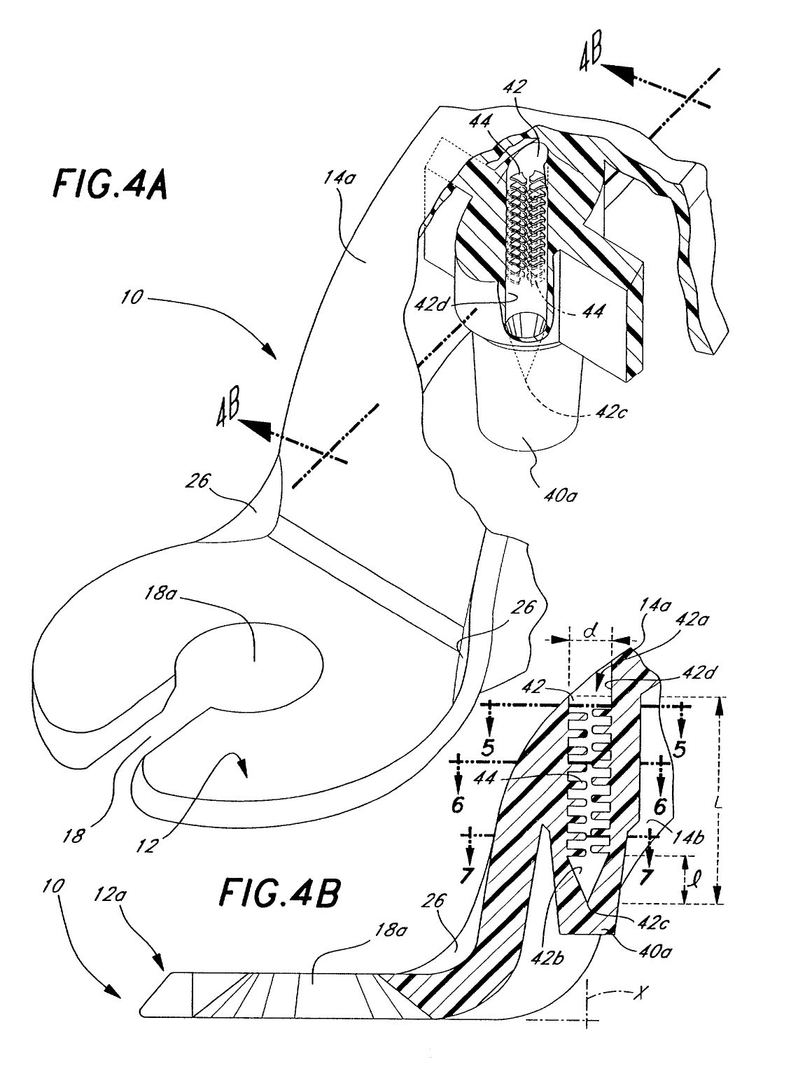

As illustrated in FIGS. 1 through 2, the tool 10 has a guard 12 attached to a handle 14. This tool 10 is made from a FDA approved polystyrene that is injected molded using conventional molding techniques to form a unitary structure about 5-6 inches long and about an inch wide. The guard 12 has a generally circular configuration with a diameter of one inch. The circumferential edge 16 of the guard 12 is beveled inwardly to allow the guard to be forced beneath a needle (FIG. 4C) that has been inserted into an implant device (not shown) when the tool 10 is being used to deaccess the needle from the implanted device. The guard 12 has a slot 18 which extends from the circumferential edge 16 inwardly and radially, terminating in an enlarged, circular opening 18a which has its center coincident with the center of the guard 12. The diameter of the circular opening 18a is about ¼ inch. The opposed sides 18b of the slot 18 are beveled inwardly.

As best shown in FIG. 2, the underside 12a of the guard 12 has a central depression in the form of a C-shaped, concave section 20. The diameter of this concave section is about ½ inch, which corresponds approximately to the outside diameter of the top portion of the implanted device. Surrounding this concave section 20 is a flat peripheral bottom, comprising a pair of approximately semi-circular sections 22a and 22b that straddle the slot 18. The depth of the concave section 20 at its center is about 3/16 inch. Optionally, indicia may be placed on the guard 12 to assist in aligning the circular opening 18a with the implanted device during accessing and deaccessing.

The handle 14 is uniquely shaped to provide a reinforced structure that prevents it from twisting or bending when used in a normal fashion. One unique characteristic of the handle 14 is a raised thumb lever 24 approximately 2½ inches from the center of the enlarged opening 18a. This thumb lever 24 serves as a marker indicating that the hand of the nurse should be behind this thumb lever. A pair of opposed, triangular, reinforcing pieces 26 at the rear of the guard 12 are integral with the handle 14 at the point where a top wall 14a of the handle 14 and opposed side walls 14b of the handle merge with the rear of the guard 12. The forward portion of the top wall 14a slopes downward from the apex 24a of the thumb lever 24 towards the guard 12, changing its direction of curvature, and then merges with the rear of the guard. The rearward portion of the top wall 14a slopes downward from the apex 24a of the thumb lever 24, then substantially flattens into an elongated portion that terminates at the end of the handle 14. The opposed side walls 14b are approximately of the same height and are substantially at a right angle to the top wall 14a. There is a rear wall 14c closing off the end of the handle 14, which is also at a substantially right angle with respect to the top wall 14a. The junction where the side walls 14b and rear wall 14c intersect with the top wall 14a are beveled, and the inside edges of the side walls and rear wall are beveled inwardly.

As shown in FIG. 2, the underside 12a of the guard 12 and the forward portion of the top wall 14a after changing its direction of curvature form a connector section 23. The front face of this connector section 23 and the underside 12 a of the guard 12 form an angle of about 80 degrees. This assists in creating a downward force applied to the guard 12 when the nurse applies a downward pressure when grasping the handle 14 and positioning her or his thumb against the thumb lever 24,

The indicia 25 on the guard 12 assist the nurse in positioning the guard in an overlying position with respect to the implanted device. With the guard in position overlying the implanted device, the nurse pushes with the thumb against the thumb lever 24, so that a substantial downward component of force is applied by the guard against the skin overlying the implanted device. As shown in FIGS. 4A and 4C, the upper portion of the guard 12, along with the skin under the guard 12 that overlies the implanted device, seats itself within the concave section 20 of the guard 12 of the implanted device, when a downward force or pressure is applied through the tool 10 to the implanted device.

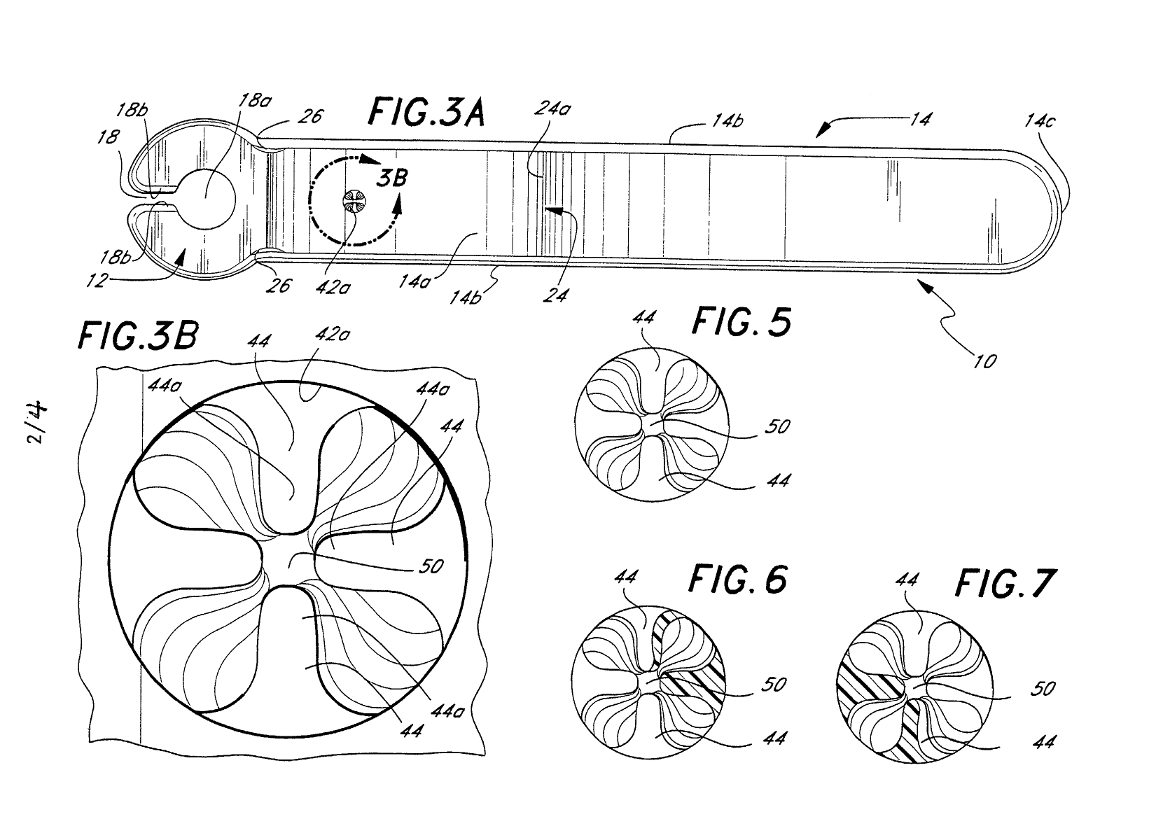

As best shown in FIG. 2, in accordance with this invention, the tool 10 has a retainer 40 for holding the needle 5 in the handle top wall 14a after being withdrawn from an implanted device. The body 40a of the retainer is integral with the underside of the top wall 14a. As best depicted in FIGS. 3B, 4A and 4B, the retainer 40 has an internal cavity 42 with an open mouth 42a in the top wall 14a of the handle 14 between the guard 12 and the thumb lever 24. This cavity 42 has a cylindrical side wall 42d terminating in a bottom section 42b that has substantially conical configuration with its apex 42c providing a constriction in which the tip of an inserted needle is lodged. The diameter d of the open mouth 42a is about 3/16 inch, the total length L of the cavity 42 is about ½ inch, and the length l of the bottom section 42b is about 1/16 inch. The longitudinal axis x of the cavity 42 is at an angle of 90 degrees with respect to the underside 22a of the guard section 12.

Projecting outward from the cylindrical side wall 42d of cavity 42 into the cavity are radial fingers 44 that overlie each other and are arranged in a helical manner along the side wall 42d. These fingers 44 are flexible. As best shown in FIG. 3B, the fingers 44 are configured in sets where each set has four fingers 44. The fingers 44 are substantially at a right angle with respect to the longitudinal axis x of the cavity 42, and they are offset with respect to each other. The sets of fingers 44 are slightly unaligned and not in exact registration as illustrated in FIG. 5-7. Each finger 44 in each set is spaced apart to provide a cross-shaped opening between the fingers in each set. The central portion 50 of each set is open, and is in registration with the open central portion of the other sets. These aligned central portions 50 provide an elongated central open space along the longitudinal axis x of the cavity 42 that is an ideal target into which the tip of the needle 5 is forced upon inserting the needle into the cavity. As depicted in FIG. 4C, the tip of the inserted needle bottoms out in the conical bottom section 42b and is lodged in the apex 42c. The fingers 44 are bent by the needle 5 downward towards the apex 42c, engaging the shank 5a of the needle 5. If the user does not hit the ideal target, the central portions 50, the fingers 44, being flexible, will bend upon engaging the tip of the needle. Consequently, the fingers 44 will still grip the shank 5a of the needle 5 upon insertion into the retainer 40.

When the needle 5 is inserted into the retainer 40, the tip of the needle preferably fits into the central open space provided by central portions 50 of the sets of fingers 44. However, if the nurse does not exactly position the tip of the needle 5 to extend into this central open space, the tip of the needle will still fit between the fingers 44 which flex to provide open space for the tip of the needle. Since there are a number of sets of fingers 44 that grasp the tip of the needle 5 along the portion of the needle extending into the retainer 40, there is little likelihood that the needle will be dislodged from the retainer, even if the tool 10 is inverted. Thus, the tool 10, with the needle captured in the retainer 40, may be discarded safely.

Second Embodiment:

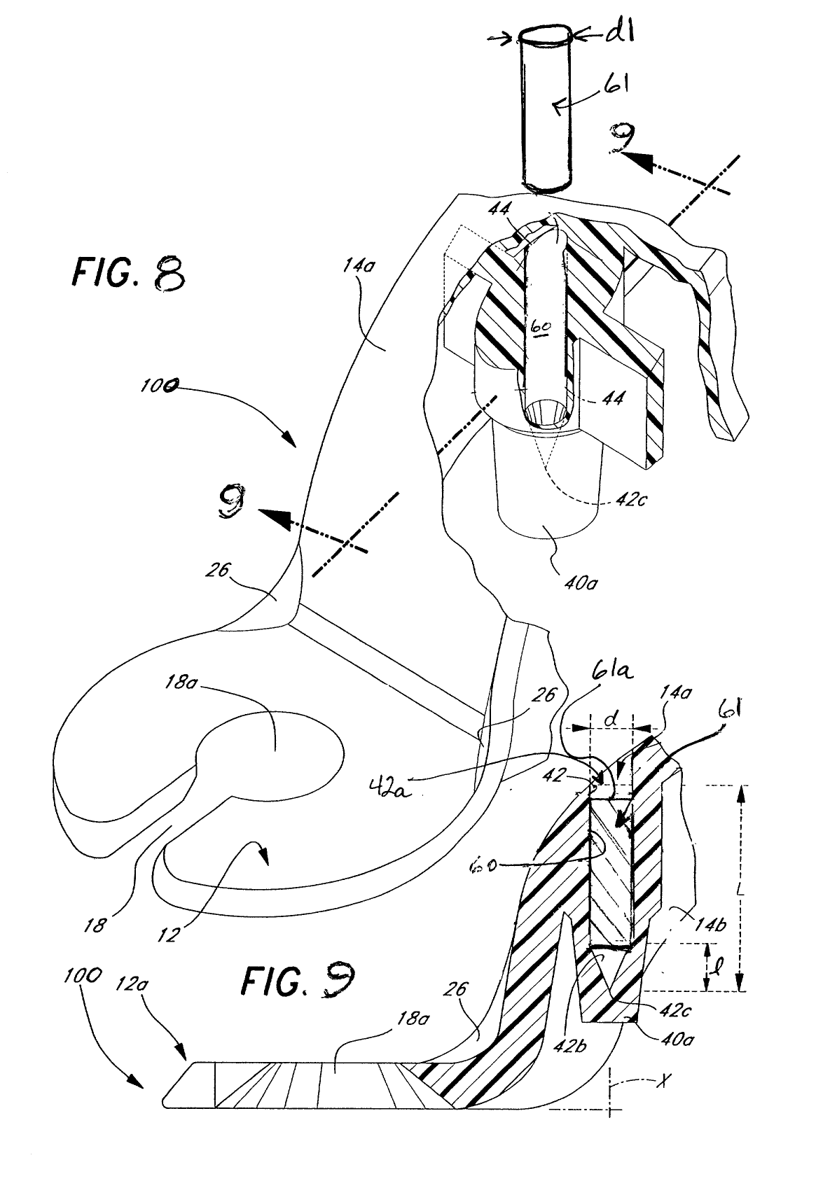

As illustrated in FIGS. 8 and 9, an alternate embodiment of this invention, the tool 100, is substantially the same as the tool 10 employing the retainer 40 with the radial fingers 44, expect the retainer 40 with the radial fingers 44 is eliminated. The retainer 40 is replaced by a cylindrically shaped, plug-type retainer 61, preferably made of an elastomeric material such as polyurethane. Preferably, this elastomeric material has a durometer hardness from about 45 to about 65. Elimination of the radial fingers 44 provides a smooth inside cylindrical wall 60 for the internal cavity 42. Preferably, the plug-type retainer 61 has length slightly less than the depth of the cavity and it is pushed deep into the cavity so that its top end 61a is recessed with respect to the open mouth 42a of the cavity in the top wall 14a of the handle 14. Preferably, the diameter dl of the plug-type retainer 61 is slightly greater than the diameter d of the open mouth 42a. Consequently, the retainer 61 fits snugly within the cavity and does not come loose after being inserted because of its resilient character. For this same reason, once a needle tip is inserted into the top end, it also tends to stay and not come loose after being inserted.

### General Procedures

Conventional aseptically clean techniques are followed in using the tools and methods of this invention, and the needle is flushed with saline and Heparin solutions to avoid clogging. Clean or sterile rubber gloves should be worn when accessing and deaccessing the needle, and all used needles are disposed of in sharps safety containers, or safely by inserting in the retainer 40 or 61, and then placed in a sharps safety container.

### SCOPE OF THE INVENTION

The above presents a description of the best mode contemplated of carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above which are fully equivalent. Consequently, it is not the intention to limit this invention to the particular embodiment disclosed. On the contrary, the intention is to cover all modifications and alternate constructions coming within the spirit and scope of the invention as generally expressed by the following claims, which particularly point out and distinctly claim the subject matter of the invention:

1. A tool adapted to be used with an implanted device for accessing and deaccessing a needle, including
an elongated body including a handle section and a guard section, and
a slot extending from an edge of the guard section into the guard section and terminating at an internal portion of the guard section,
said handle section having therein a retainer member made of elastomeric material adapted to receive the tip of a needle withdrawn from an implanted device.

2. The tool of claim 1 where the retainer member has a top end and is seated within an elongated cavity with an open mouth so that the top end is recessed with respect to the open mouth.

3. The tool of claim 1 where the elongated cavity that is at an angle of substantially 90 degrees with respect to the guard section.

4. The tool of claim 1 where the guard has an underside with a concave section having a substantially circular rim and the slot that extends radially outward from the center of the concave section to the edge of said guard section.

5. The tool of claim 4 where the concave section has a depth of from 1/16 to 3/16 inch.

6. The tool of claim 4 where the rim has a diameter of from 1/16 to ½ inch.

7. The tool of claim 4 where the guard has an area of 1.0 square inches or more.

8. The tool of claim 4 where the handle has a length of 3 inches or more.

9. The tool of claim 4 where the slot terminates in the guard section at in an enlarged open portion.

10. The tool of claim 9 where the enlarged open portion has an area in excess of 0.0036 square inch.

11. The tool of claim 1 where the slot has a width of from 1/16 to ¼ inch.

12. The tool of claim 1 where the slot and the handle section are in substantial alignment with each other, both lying along a common longitudinal axis.

13. The tool of claim 1 said handle section has a marker indicating that a hand of a nurse should be behind said marker, said marker being located to position the hand of the nurse at least 2.5 inches away from the needle when the tool is used to access or deaccess a needle in an implanted device, and where the marker comprises a raised thumb lever.

14. A tool adapted to be used with an implanted device for accessing and deaccessing a needle, including
an elongated body including a handle section and a guard section, and
a slot extending from an edge of the guard section into the guard section and terminating at an internal portion of the guard section,
said handle section having a top surface within a cavity therein having an open mouth in which is seated with an elastomeric, plug-type retainer member that fits snugly within the cavity and below the open mouth of the cavity to provided a recessed top end adapted to receive the tip of a needle withdrawn from an implanted device.

15. The tool of claim 14 where the elastomeric, plug-type retainer is made of a material that has a durometer hardness from 45 to 65.

16. A method for manually removing a needle from a medical device implanted under the skin of a patient, including
grasping with one hand a handle section of a tool having a guard section, and an slot extending from an edge of the guard section into the guard section and terminating at an internal portion of the guard section, said handle section having a top surface with an elastomic, plug-type retainer member therein into which the tip of a needle may be inserted,
sliding the slot along the needle to bring the guard section into an overlying relationship with the implanted medical device,
maintaining said one hand a minimum distance away from the needle of at least 2.5 inches,
with the other hand withdrawing said needle free of the implanted medical device, and
inserting said withdrawn needle into the retainer.
